(19) United States
(12) Plant Patent Application Publication
(10) Pub. No.: US 2010/0293685 P1
(43) Pub. Date: Nov. 18, 2010

(54) Marguerite with variegated leaf foliage

(50) ;

(76) Inventor: Dale Edward Layton, (US)

Correspondence Address: PENINSULA IP GROUP, 26150 BUCKS RUN, CORRAL DE TIERRA, CA, 93908, US

(21) Appl. No.: 12/454,216

(22) Filed: May 13, 2009

(51) Int. Cl. A01H 5/00

(52) U.S. Cl. PLT/286

(57) ABSTRACT

Disclosed herein is a new and distinct Marguerite plant characterized by the following characteristic: variegated leaf foliage.

### BACKGROUND OF THE INVENTION

1. Field of the Invention

The present invention relates to all marguerite plants capable of having variegated leaf foliage. Typically marguerites occur in either of two plants varieties, namely Chrysanthemum frutescens and C. leucanthemum. These plants resemble the common American daisy, also known as Paris daisy.

2. Background of the Invention

Marguerites have long been a popular garden staple. Marguerites have been grown for over 200 years and it is reported that there are over 80 types currently available. Marguerites are typically hybrids and come in wide varieties from bushy, shrubby type growth that can be left in a shaggy type shape to topiarised and manicured.

As set forth in the Plant encyclopedia—BHG.com, the “total absence of plastid pigments causes a sector of a leaf or stem to have white patches. This condition is termed variegation (Metrosideros). Variegation is produced when there is a cell mutation (cytological chimera), and all cells produced from that mutant mother cell lack the pigments, either because plastids are not present or the plastid cannot complete the manufacture of the pigment apparatus. White, therefore, is where color is missing. The zones where chloroplasts are not present are zones where no photosynthesis will occur, hence a variegated leaf has a lowered potential to fix carbon dioxide into sugars, and as a consequence, a variegated plant also tends to grow more slowly.

“Variegated leaves occur rarely in nature but are extremely common among indoor and outdoor ornamentals, where they have been saved as horticultural oddities. Species with variegated individuals are sometimes found in the understory of tropical rain forest, and this habitat is the source of a number of variegated house plants. The appearance of variegation in the tropical forest understory, if not simply by accident, has not been given a plausible explanation.

“Some variegated ornamentals have only a fringe of white around the leaf (Example: Pittosporum), sometimes irregular dots (Examples: a bromeliad and Begonia) and spots (Examples: Dieffenbachia, an aloe, and an orchid), sometimes broad panels of white (Examples: a bromeliad, shell ginger, Pisonia, and a fig), and even an occasional leaf that is totally achlorophyllous (white; Example: Hedera helix). Among monocotyledons, strips of stem internode may also be achlorophyllous (Example: variegated sugar cane). On a single plant, you can observe leaves with all different degrees of variegation, but a plant may also produce individual leaves or entire shoots that are all green (Examples: Coprosma, Abutilon, Euonymus, and Bougainvillea), and which did not involve mother cells with mutant plastids. On the same plant, totally green leaves tend to be much larger than the variegated ones. Some very colorful variegated horticultural forms occur when the leaf cells can produce vacuolar pigments, such as anthocyanins, which then produce patches of pink where chlorophyll is absent (Example: Acalypha).

“Out of the tropics have arisen some very unusual variegated forms (Example: a gesneriad). One of the most photographed is a species of Calathea, which has patches of green, surrounded by white tissue resembling a set of leaves within a single leaf blade.

Some people may dismiss variegation as an uninteresting series of mutant forms, rare in nature, but there are some plants living in full-sun habitats that possess variegated leaves. “Examples are certain species of clover (Trifolium), possessing a V-shaped clear mark on each leaflet. Among tropical epiphytes are numerous species of bromeliads and certain orchids. Typically, variegated individuals can persist in nature via cloning, i.e., vegetative propagation by forming new plants from buds, as in bromeliads, orchids, aloes, and agaves, rather than via seeds, which would necessarily be a rare event.

“Many leaves have white or nongreen veins, causing the leaf to appear variegated (Examples: an aroid, Sanchezia, striped inch plant, and milk thistle). The white vein is caused by the absence of chlorophyll-bearing cells above or below the conducting tissues of the vein. Most commonly, no stomates are found directly on a vein, so the cells, incapable of receiving adequate carbon dioxide, are not equipped for photosynthesis. Leaves with white veins should not be classified as variegated, although they are sometimes also grown as horticultural oddities.”

As stated by R. Daniel Lineberger in “Orgin, Development, and Propagation of Chimeras, “A plant is said to be a chimera when cells of more than one genotype (genetic makeup) are found growing adjacent in the tissues of that plant. Variegated plants are perhaps the most common types of chimeras, and certainly the most convenient example to use in presenting the basic concept.

“The apical meristem of a shoot is the location where most of the cells which produce the plant body are formed. Cell division occurs at a very rapid rate in an actively growing shoot and these cells in turn elongate or expand resulting in growth in length of the shoot.

“Chimeras arise when a cell undergoes mutation. This mutation may be spontaneous or it may be induced by irradiation or treatment with chemical mutagens. If the cell which mutates is located near the crest of the apical dome, then all other cells which are produced by division from it will also be the mutated type. The result will be cells of different genotypes growing adjacent in a plant tissue, the definition of a chimera.

“If the location of the cell at the time of mutation is in a region where little further cell division will occur, then the likelihood of detecting this mutation by visual inspection of the whole plant is low. Furthermore, if the mutation results in a genotype which is not very different morphologically from the rest of the plant, then the likelihood of identifying the plant as a chimera is also low. A mutation which results in colorless rather than green cells (variegation) is easily detectable, whereas a mutation which results in greater sugar accumulation in the cells would not be observed.

“Careful observation of the pattern of coloration in variegated dicot leaves allows one to partially deduce the nature of the chimeral layers in the shoot apex. A complete characterization of the nature of all three layers cannot be made macroscopically since most of the cells of the epidermal layer of dicot leaves do not produce chloroplasts even if the genetic information for chloroplast development is present.

“Stable periclinal chimeras have resulted in plant sports with unique ornamental features. The ability to propagate these sports in a true-to-type fashion depends upon the use of a propagation technique which utilizes lateral buds. The layered organization of the apical meristem which is characteristic for a periclinal chimera is maintained in lateral meristems, but is usually lost during the differentiation of adventitious shoots. Plant tissue culture methodology enables the separation of chimeral genotypes in some species, facilitating the study of the separated component genotypes. This advantage of tissue culture may be a liability in systems which proliferate in vitro through adventitious shoot formation, since the propagation of off-type plants may occur.

What has never been developed or found is a variegated leaf foliage in a marguerite plant. The invention herein discloses such a new variety, which is both stable and reproducible.

### SUMMARY OF THE INVENTION

The new variety of Marguerite is created by taking cuttings from a SPORT. The cutting is treated with root tone and then placed under lights in a potting mixture of vermiculite. The lighting and container size as well as the temperature and water conditions are adjusted for plant growth.

The new variety is bred to form
a. strong and disease resistance vegetation;
b. bushy and thick foliage;
c. blue/green color;
d. rapid, but not excessive growth rate;
e. short stems;
f. thick stems;
g. desirable flower

The new variety meets the above criteria and in addition produces a compact plant, which roots quickly and flowers consistent with other well growing Marguerites.

### DETAILED DESCRIPTION OF THE INVENTION

In the spring, seeds that were planted earlier begin to sprout. The seedlings are placed in a container. Upon further growth, the seedlings form fully grown plants and then are examined. Using a Sport, Applicant found the beginnings of variegation. Upon further cuttings and growth, Applicant developed his variegated variety.

From the fully grown plant, Applicant takes cuttings having the desired mutation of variegation. Those cuttings are treated with root tone and planted in vermiculite.

The containers having the seedlings are 1 ounce cups. It has been found that clear cups work best to be able to view the rootings.

In the containers are filled with 1 ounce of vermiculite and ¾ of an ounce of water.

Proper plant lighting is essential to proper growth. An plant aquarium light is used for 12-14 hour duration each day to provide optimum growing conditions.

The plants while maturing are they are kept in 12″-14″ closed containers.

The temperature is kept between 50 degrees and 60 degree F.

After rooting, about 98%, the plants are transpotted to an 8 ounce styro-foam cup. The plants are then provided with heavy shade, heavy water. In about 10 days, the plants are transplanted to natural sunlight in about 1 gallon drums. For the next 2½ months to 4 months, the plants are allowed to grow and develop into mature adults.

### DESCRIPTION OF FIGURES

FIG. 1 is a top view of a cutting of the Marguerite in accordance with this invention. As seen, the flowering portion is unaffected by the variegated foliage.

FIG. 2 is an enlarged picture of the variegated foliage in accordance with this invention.

FIG. 3 illustrates a top view of the plant in accordance with this invention. As seen, the variegated portion is visible through the foliage and the growth and makes a most distinctive appearance.

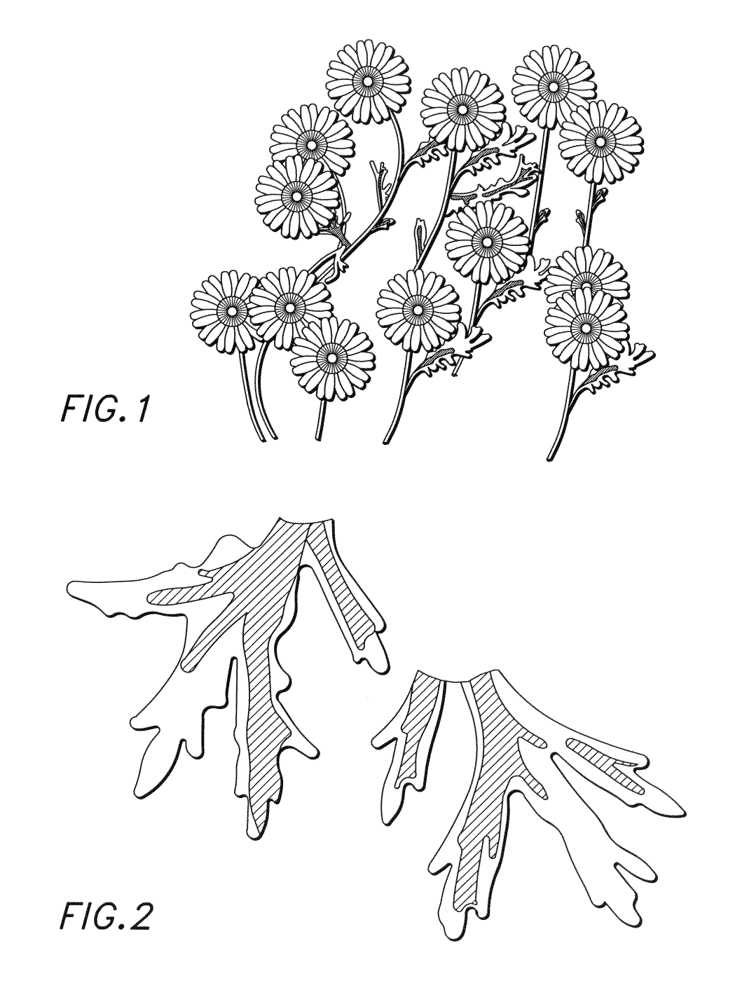

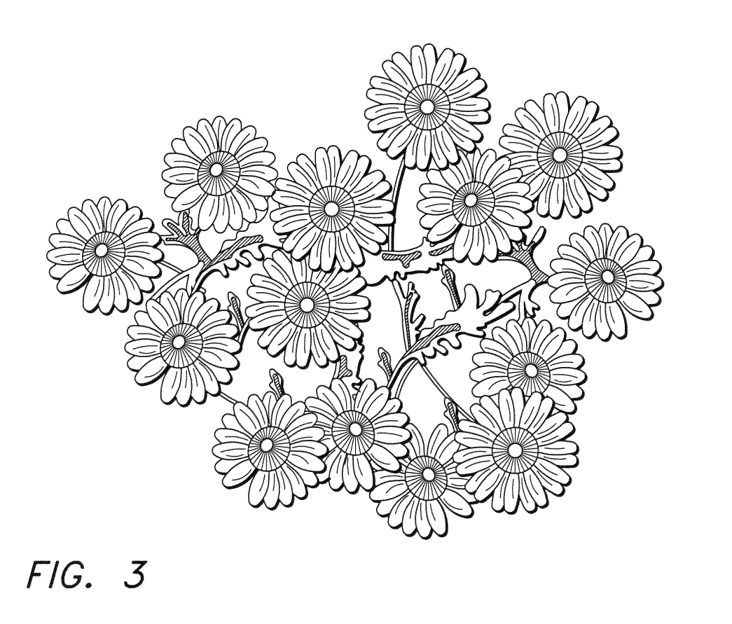

### DESCRIPTION OF PICTURES

Pictures 1 through 4 are included. Each picture represents a different view of the variegated Marguerite.

Thus, the invention is to be limited only by the claims as set forth below.

What is claimed is:

1. A new and distinct Marguerite plant characterized by the following characteristic:
variegated leaf foliage.
